(19) United States
(12) Patent Application Publication
(10) Pub. No.: US 2014/0052202 A1
(43) Pub. Date: Feb. 20, 2014

(54) MEDICAL DEVICE WITH SCREEN, AND DOOR COVERING THE SCREEN

(71) Applicant: John C. Daynes, Redmond, WA (US)

(72) Inventor: John C. Daynes, Redmond, WA (US)

(73) Assignee: PHYSIO-CONTROL, INC., Redmond, WA (US)

(21) Appl. No.: 13/589,911

(22) Filed: Aug. 20, 2012

(51) Int. Cl. A61N 1/39

(52) U.S. Cl. 607/5

(57) ABSTRACT

Medical devices and methods of operating medical devices that treat and monitor patients include a housing and a module located within the housing. The module is configured to perform the treating and monitoring parameters of the patient. A screen is also attached to the housing. The screen is viewable by the user outside of the housing. The medical device also includes a door that is coupled with the housing. The door is movable between a closed position that covers at least some portion of the coverable portion of the screen so as to prevent the coverable portion of the screen from being viewed and an open position that does not cover the coverable portion of the screen.

### CROSS-REFERENCE TO RELATED PATENT APPLICATIONS

This patent application may be found to be related to U.S. patent application Ser. No. [SER_NO_OF_{—}40037], filed on the same day as the instant application, with the same inventor and on behalf of the same assignee.

### FIELD

This invention generally relates to medical devices, including defibrillators.

### BACKGROUND

In humans, the heart beats to sustain life. In normal operation, it pumps blood through the various parts of the body. More particularly, the various chamber of the heart contract and expand in a periodic and coordinated fashion, which causes the blood to be pumped regularly. More specifically, the right atrium sends deoxygenated blood into the right ventricle. The right ventricle pumps the blood to the lungs, where it becomes oxygenated, and from where it returns to the left atrium. The left atrium pumps the oxygenated blood to the left ventricle. The left ventricle, then, expels the blood, forcing it to circulate to the various parts of the body.

The heart chambers pump because of the heart's electrical control system. More particularly, the sinoatrial (SA) node generates an electrical impulse, which generates further electrical signals. These further signals cause the above-described contractions of the various chambers in the heart, in the correct sequence. The electrical pattern created by the sinoatrial (SA) node is called a sinus rhythm.

Sometimes, however, the electrical control system of the heart malfunctions, which can cause the heart to beat irregularly, or not at all. The cardiac rhythm is then generally called an arrhythmia. Arrhythmias may be caused by electrical activity from locations in the heart other than the SA node. Some types of arrhythmia may result in inadequate blood flow, thus reducing the amount of blood pumped to the various parts of the body. Some arrhythmias may even result in a Sudden Cardiac Arrest (SCA). In a SCA, the heart fails to pump blood effectively, and, if not treated, death can occur. In fact, it is estimated that SCA results in more than 250,000 deaths per year in the United States alone. Further, a SCA may result from a condition other than an arrhythmia.

One type of arrhythmia associated with SCA is known as Ventricular Fibrillation (VF). VF is a type of malfunction where the ventricles make rapid, uncoordinated movements, instead of the normal contractions. When that happens, the heart does not pump enough blood to deliver enough oxygen to the vital organs. The person's condition will deteriorate rapidly and, if not reversed in time, they will die soon, e.g. within ten minutes.

Ventricular Fibrillation can often be reversed using a life-saving device called a defibrillator. A defibrillator, if applied properly, can administer an electrical shock to the heart. The shock may terminate the VF, thus giving the heart the opportunity to resume pumping blood. If VF is not terminated, the shock may be repeated, often at escalating energies.

A challenge with defibrillation is that the electrical shock must be administered very soon after the onset of VF. There is not much time: the survival rate of persons suffering from VF decreases by about 10% for each minute the administration of a defibrillation shock is delayed. After about 10 minutes the rate of survival for SCA victims averages less than 2%.

The challenge of defibrillating early after the onset of VF is being met in a number of ways. First, for some people who are considered to be at a higher risk of VF or other heart arrythmias, an Implantable Cardioverter Defibrillator (ICD) can be implanted surgically. An ICD can monitor the person's heart, and administer an electrical shock as needed. As such, an ICD reduces the need to have the higher-risk person be monitored constantly by medical personnel.

Regardless, VF can occur unpredictably, even to a person who is not considered at risk. As such, VF can be experienced by many people who lack the benefit of ICD therapy. When VF occurs to a person who does not have an ICD, they collapse, because blood flow has stopped. They should receive therapy quickly.

For a VF victim without an ICD, a different type of defibrillator can be used, which is called an external defibrillator. External defibrillators have been made portable, so they can be brought to a potential VF victim quickly enough to revive them.

During VF, the person's condition deteriorates, because the blood is not flowing to the brain, heart, lungs, and other organs. Blood flow must be restored, if resuscitation attempts are to be successful.

Cardiopulmonary Resuscitation (CPR) is one method of forcing blood flow in a person experiencing cardiac arrest. In addition, CPR is the primary recommended treatment for some patients with some kinds of non-VF cardiac arrest, such as asystole and pulseless electrical activity (PEA). CPR is a combination of techniques that include chest compressions to force blood circulation, and rescue breathing to force respiration.

Properly administered CPR provides oxygenated blood to critical organs of a person in cardiac arrest, thereby minimizing the deterioration that would otherwise occur. As such, CPR can be beneficial for persons experiencing VF, because it slows the deterioration that would otherwise occur while a defibrillator is being retrieved. Indeed, for patients with an extended down-time, survival rates are higher if CPR is administered prior to defibrillation.

Advanced medical devices can actually coach a rescuer who performs CPR. For example, a medical device can issue instructions, and even prompts, for the rescuer to perform CPR more effectively.

A reality is that possible users of defibrillators and other medical devices could have different skill levels. In some instances, an experienced person might want to use all the controls of a medical device, while an inexperienced person might be overwhelmed by them. In the case of a defibrillator, the potential disparity can be a different mode of use, which has been addressed in co-owned U.S. Pat. No. 6,754,526, for example. Less experienced users benefit from some functions and features of the defibrillators being automatic rather than manual. More experienced users are likely more familiar with the defibrillators and prefer to have manual control over all or most of the functions and features of the defibrillator. Alternatively, less experienced users are likely more comfortable with and can provide more effective treatment and monitoring to patients if only essential features and functions of the defibrillators are available for use. More experienced users and medical professionals are specially trained to use the more advanced features and functions of the defibrillators and are likely to want access to the full range of the defibrillators capabilities when treating and/or monitoring a patient.

Defibrillators are very useful when they can be operated by both experienced and less or inexperienced users. Such a multi-level defibrillator is cost-efficient and provides greater availability to help treat and monitor patients needing assistance. However, providing multi-level defibrillators is complicated because they need to have the ability to change between modes and/or offer different functions and features easily and quickly. Further, space is usually limited on defibrillators and providing multiple interfaces is challenging to fit into the limited space. Embodiments of the invention address these and other limitations of the prior art.

### BRIEF SUMMARY

The present description gives instances of medical devices, systems, software and methods, the use of which may help overcome problems and limitations of the prior art.

In one embodiment, medical devices for a user to one of treat a patient and monitor a parameter of a patient include a housing, a module located in the housing, a screen attached to the housing, and a door that is coupled to the housing. The module treats and/or monitors the patient parameter(s). The screen is viewable by the user outside of the housing. The door is movable between a closed position that covers at least a coverable portion of the screen to prevent it from being viewed and an open position that does not cover the coverable portion of the screen.

In another embodiment, a method of treating a patient and/or monitoring a parameter of the patient using a medical device includes causing the door to move to a closed position that covers at least a coverable portion of a screen attached to a housing of the medical device to prevent the coverable portion of the screen from being viewed by the user and causing the door to move to an open position that does not cover the coverable portion of the screen. The medical device includes any medical devices disclosed herein, such as the medical device described above.

These and other features and advantages of this description will become more readily apparent from the following Detailed Description, which proceeds with reference to the drawings, in which:

### BRIEF DESCRIPTION OF THE DRAWINGS

FIG. 1 is a diagram of a scene where an external defibrillator is used to save the life of a person according to embodiments.

FIG. 2 is a table listing two main types of the external defibrillator shown in FIG. 1, and who they might be used by.

FIG. 3 is a diagram showing components of an external defibrillator, such as the one shown in FIG. 1, which is made according to embodiments.

FIG. 4 is a block diagram of a medical device with a door movable between a closed position and an open position according to embodiments.

FIG. 5 is a block diagram of the screen of a medical device when the door is in a closed position and an open position in accordance with embodiments.

FIG. 6 is a block diagram of a screen of a medical device when the door is in the closed position and the open position according to embodiments.

FIG. 7 is a block diagram of a medical device and door with a defibrillation module according to embodiments.

FIG. 8 is a block diagram of a medical device and door that controls the operating state of the medical device depending on the door's position according to embodiments.

FIG. 9 is a block diagram of the unnecessarily and coverable portions of the screen of the medical device according to embodiments.

FIG. 10 is a block diagram of another embodiment of the unnecessarily and coverable portions of the screen of the medical device in accordance with embodiments.

FIG. 11A shows an embodiment of the medical device with the door in the closed position.

FIG. 11B shows an embodiment of the medical device shown in FIG. 11A with the door in the open position.

FIG. 12 is a flowchart illustrating methods according to embodiments.

### DETAILED DESCRIPTION

As has been mentioned, the present description is about medical devices with user interfaces that are implemented with a door. In some embodiments, the door can be shut and cover a portion of a screen of the device. Embodiments are now described in more detail.

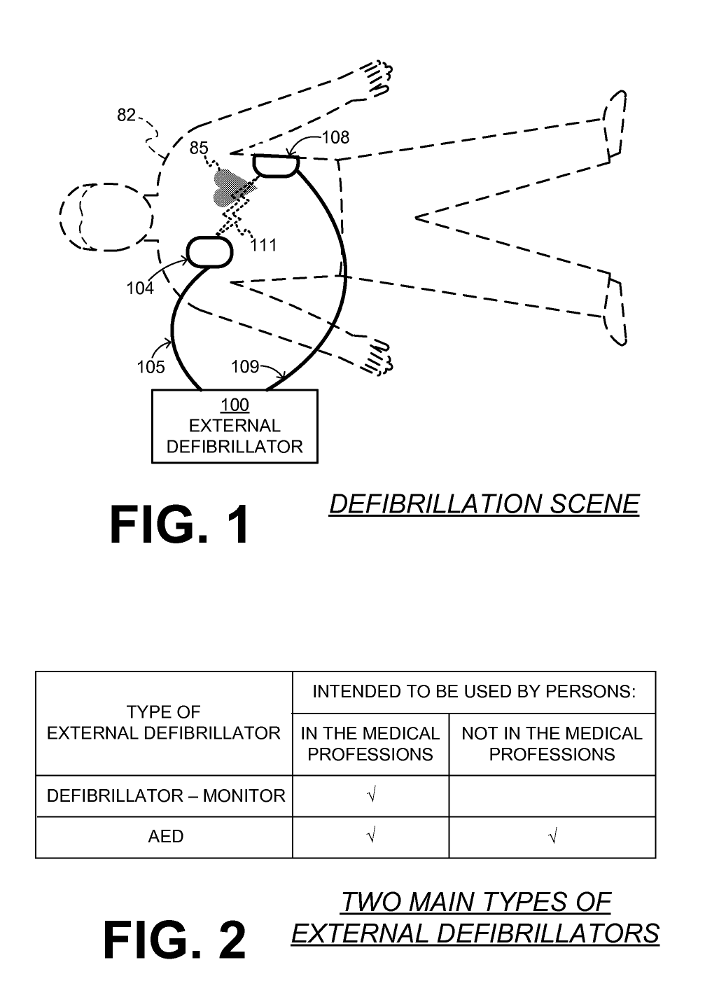

FIG. 1 is a diagram of a defibrillation scene. A person 82 is lying on their back. Person 82 could be a patient in a hospital, or someone found unconscious, and then turned to be on their back. Person 82 is experiencing a condition in their heart 85, which could be Ventricular Fibrillation (VF).

A portable external defibrillator 100 has been brought close to person 82. At least two defibrillation electrodes 104, 108 are usually provided with external defibrillator 100, and are sometimes called electrodes 104, 108. Electrodes 104, 108 are coupled with external defibrillator 100 via respective electrode leads 105, 109. A rescuer (not shown) has attached electrodes 104, 108 to the skin of person 82. Defibrillator 100 is administering, via electrodes 104, 108, a brief, strong electric pulse 111 through the body of person 82. Pulse 111, also known as a defibrillation shock, goes also through heart 85, in an attempt to restart it, for saving the life of person 82.

Defibrillator 100 can be one of different types, each with different sets of features and capabilities. The set of capabilities of defibrillator 100 is determined by planning who would use it, and what training they would be likely to have. Examples are now described.

FIG. 2 is a table listing two main types of external defibrillators, and who they are primarily intended to be used by. A first type of defibrillator 100 is generally called a defibrillator-monitor, because it is typically formed as a single unit in combination with a patient monitor. A defibrillator-monitor is sometimes called monitor-defibrillator. A defibrillator-monitor is intended to be used by persons in the medical professions, such as doctors, nurses, paramedics, emergency medical technicians, etc. Such a defibrillator-monitor is intended to be used in a pre-hospital or hospital scenario.

As a defibrillator, the device can be one of different varieties, or even versatile enough to be able to switch among different modes that individually correspond to the varieties. One variety is that of an automated defibrillator, which can determine whether a shock is needed and, if so, charge to a predetermined energy level and instruct the user to administer the shock. Another variety is that of a manual defibrillator, where the user determines the need and controls administering the shock.

As a patient monitor, the device has features additional to what is minimally needed for mere operation as a defibrillator. These features can be for monitoring physiological indicators of a person in an emergency scenario. These physiological indicators are typically monitored as signals. For example, these signals can include a person's full ECG (electrocardiogram) signals, or impedance between two electrodes. Additionally, these signals can be about the person's temperature, non-invasive blood pressure (NIBP), arterial oxygen saturation/pulse oximetry (SpO2), the concentration or partial pressure of carbon dioxide in the respiratory gases, which is also known as capnography, and so on. These signals can be further stored and/or transmitted as patient data.

A second type of external defibrillator 100 is generally called an AED, which stands for “Automated External Defibrillator”. An AED typically makes the shock/no shock determination by itself, automatically. Indeed, it can sense enough physiological conditions of the person 82 via only the shown defibrillation electrodes 104, 108 of FIG. 1. In its present embodiments, an AED can either administer the shock automatically, or instruct the user to do so, e.g. by pushing a button. Being of a much simpler construction, an AED typically costs much less than a defibrillator-monitor. As such, it makes sense for a hospital, for example, to deploy AEDs at its various floors, in case the more expensive defibrillator-monitor is more critically being deployed at an Intensive Care Unit, and so on.

AEDs, however, can also be used by people who are not in the medical profession. More particularly, an AED can be used by many professional first responders, such as policemen, firemen, etc. Even a person with only first-aid training can use one. And AEDs increasingly can supply instructions to whoever is using them.

AEDs are thus particularly useful, because it is so critical to respond quickly, when a person suffers from VF. Indeed, the people who will first reach the VF sufferer may not be in the medical professions.

Increasing awareness has resulted in AEDs being deployed in public or semi-public spaces, so that even a member of the public can use one, if they have obtained first aid and CPR/AED training on their own initiative. This way, defibrillation can be administered soon enough after the onset of VF, to hopefully be effective in rescuing the person.

There are additional types of external defibrillators, which are not listed in FIG. 2. For example, a hybrid defibrillator can have aspects of an AED, and also of a defibrillator-monitor. A usual such aspect is additional ECG monitoring capability.

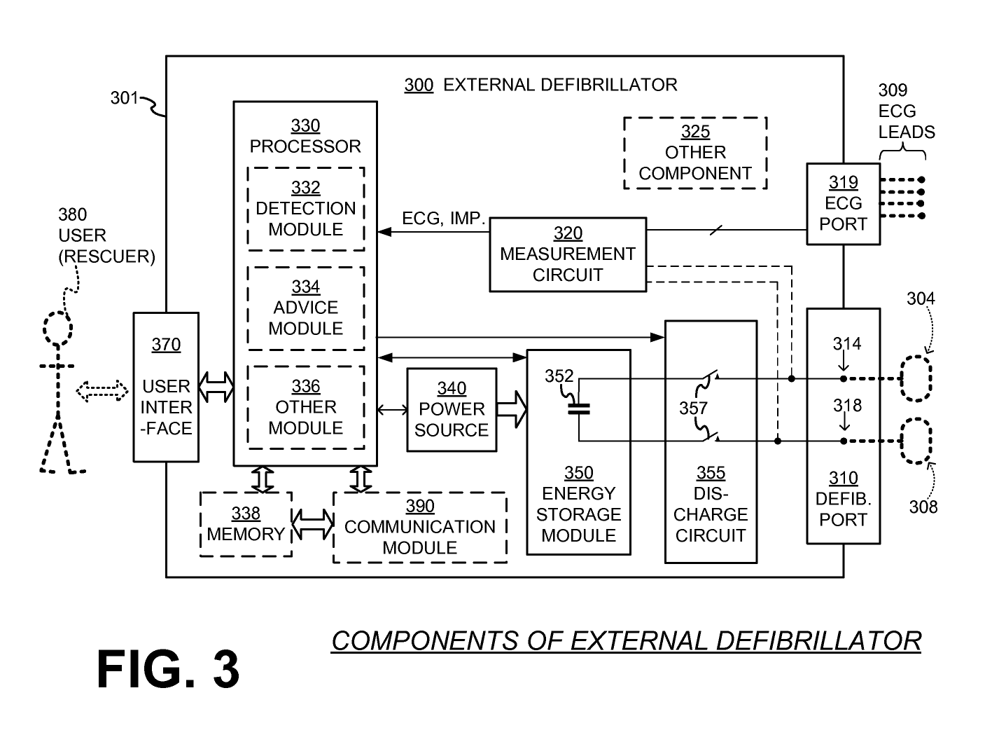

FIG. 3 is a diagram showing components of an external defibrillator 300 made according to embodiments. These components can be, for example, in external defibrillator 100 of FIG. 1. Plus, these components of FIG. 3 can be provided in a housing 301, which is also known as casing 301.

External defibrillator 300 is intended for use by a user 380, who would be the rescuer. Defibrillator 300 typically includes a defibrillation port 310, such as a socket in housing 301. Defibrillation port 310 includes nodes 314, 318. Defibrillation electrodes 304, 308, which can be similar to electrodes 104, 108, can be plugged in defibrillation port 310, so as to make electrical contact with nodes 314, 318, respectively. It is also possible that electrodes can be connected continuously to defibrillation port 310, etc. Either way, defibrillation port 310 can be used for guiding via electrodes to person 82 an electrical charge that has been stored in defibrillator 300, as will be seen later in this document.

If defibrillator 300 is actually a defibrillator-monitor, as was described with reference to FIG. 2, then it will typically also have an ECG port 319 in housing 301, for plugging in ECG leads 309. ECG leads 309 can help sense an ECG signal, e.g. a 12-lead signal, or from a different number of leads. Moreover, a defibrillator-monitor could have additional ports (not shown), and another component 325 for the above described additional features, such as patient signals.

Defibrillator 300 also includes a measurement circuit 320. Measurement circuit 320 receives physiological signals from ECG port 319, and also from other ports, if provided. These physiological signals are sensed, and information about them is rendered by circuit 320 as data, or other signals, etc.

If defibrillator 300 is actually an AED, it may lack ECG port 319. Measurement circuit 320 can obtain physiological signals through nodes 314, 318 instead, when defibrillation electrodes 304, 308 are attached to person 82. In these cases, a person's ECG signal can be sensed as a voltage difference between electrodes 304, 308. Plus, impedance between electrodes 304, 308 can be sensed for detecting, among other things, whether these electrodes 304, 308 have been inadvertently disconnected from the person.

Defibrillator 300 also includes a processor 330. Processor 330 may be implemented in any number of ways. Such ways include, by way of example and not of limitation, digital and/or analog processors such as microprocessors and digital-signal processors (DSPs); controllers such as microcontrollers; software running in a machine; programmable circuits such as Field Programmable Gate Arrays (FPGAs), Field-Programmable Analog Arrays (FPAAs), Programmable Logic Devices (PLDs), Application Specific Integrated Circuits (ASICs), any combination of one or more of these, and so on.

Processor 330 can be considered to have a number of modules. One such module can be a detection module 332, which senses outputs of measurement circuit 320. Detection module 332 can include a VF detector. Thus, the person's sensed ECG can be used to determine whether the person is experiencing VF.

Another such module in processor 330 can be an advice module 334, which arrives at advice based on outputs of detection module 332. Advice module 334 can include a Shock Advisory Algorithm, implement decision rules, and so on. The advice can be to shock, to not shock, to administer other forms of therapy, and so on. If the advice is to shock, some external defibrillator embodiments merely report that to the user, and prompt them to do it. Other embodiments further execute the advice, by administering the shock. If the advice is to administer CPR, defibrillator 300 may further issue prompts for it, and so on.

Processor 330 can include additional modules, such as module 336, for other functions. In addition, if other component 325 is indeed provided, it may be operated in part by processor 330, etc.

Defibrillator 300 optionally further includes a memory 338, which can work together with processor 330. Memory 338 may be implemented in any number of ways. Such ways include, by way of example and not of limitation, nonvolatile memories (NVM), read-only memories (ROM), random access memories (RAM), any combination of these, and so on. Memory 338, if provided, can include programs for processor 330, and so on. The programs can be operational for the inherent needs of processor 330, and can also include protocols and ways that decisions can be made by advice module 334. In addition, memory 338 can store prompts for user 380, etc. Moreover, memory 338 can store patient data.

Defibrillator 300 may also include a power source 340. To enable portability of defibrillator 300, power source 340 typically includes a battery. Such a battery is typically implemented as a battery pack, which can be rechargeable or not. Sometimes, a combination is used, of rechargeable and non-rechargeable battery packs. Other embodiments of power source 340 can include AC power override, for where AC power will be available, and so on. In some embodiments, power source 340 is controlled by processor 330.

Defibrillator 300 additionally includes an energy storage module 350. Module 350 is where some electrical energy is stored, when preparing it for sudden discharge to administer a shock. Module 350 can be charged from power source 340 to the right amount of energy, as controlled by processor 330. In typical implementations, module 350 includes one or more capacitors 352, and so on.

Defibrillator 300 moreover includes a discharge circuit 355. Circuit 355 can be controlled to permit the energy stored in module 350 to be discharged to nodes 314, 318, and thus also to defibrillation electrodes 304, 308. Circuit 355 can include one or more switches 357. Those can be made in a number of ways, such as by an H-bridge, and so on.

Defibrillator 300 further includes a user interface 370 for user 380. User interface 370 can be made in any number of ways. For example, interface 370 may include a screen, to display what is detected and measured, provide visual feedback to the rescuer for their resuscitation attempts, and so on. Interface 370 may also include a speaker, to issue voice prompts, etc. Interface 370 may additionally include various controls, such as pushbuttons, keyboards, and so on. In addition, discharge circuit 355 can be controlled by processor 330, or directly by user 380 via user interface 370, and so on.

Defibrillator 300 can optionally include other components. For example, a communication module 390 may be provided for communicating with other machines. Such communication can be performed wirelessly, or via wire, or by infrared communication, and so on. This way, data can be communicated, such as patient data, incident information, therapy attempted, CPR performance, and so on.

A feature of a defibrillator can be CPR-prompting. Prompts are issued to the user, visual or by sound, so that the user can administer CPR. Examples are taught in U.S. Pat. No. 6,334,070 and U.S. Pat. No. 6,356,785, which are herein incorporated by reference in their entirety. Other visual and audio prompts may be used, if desired.

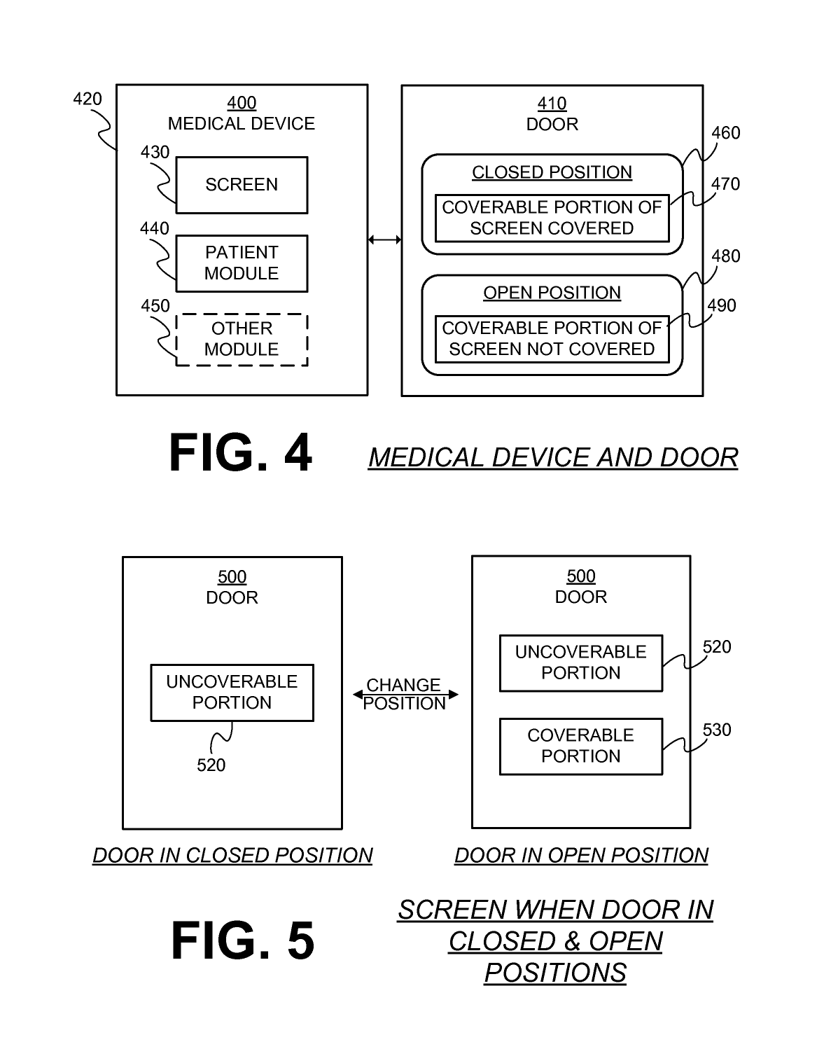

FIG. 4 illustrates a medical device 400 that is designed for a user to treat a patient, monitor parameters of a patient, or both. Treatment of a patient can include administering medical care, such as defibrillation therapy and the like. Monitoring parameters of the patient can include monitoring the patient's pulse, heart rate, blood pressure, breathing, and the like. In FIG. 4, the medical device includes a housing 420, a screen 430 attached to the housing 420, and a patient module 440 located within the housing 420. The screen 430 is viewable by the user outside of the housing. The patient module 440 provides the ability to both treat and/or monitor parameters of the patient. Optionally, one or more other modules 450 may also be located within the housing to perform other desired functions such as defibrillation, pacing, or interacting with patient data such as analyzing, storing, or transferring the data, for example.

The medical device 400 also includes a door 410 that is coupled to the housing 420. The door 410 is movable between a closed position 460 that covers at least a coverable portion of the screen 470 to prevent it from being viewed by the user and an open position 480 that does not cover the coverable portion of the screen 490. The door 410 can be moved between the closed position 460 and the open position 480 by being rotated or slid with respect to the housing 420. The door 410 can be moved in any other suitable manner with respect to the housing 420.

FIG. 5 shows the door 500 changing positions between the closed position and the open position. When the door 500 is in the closed position, the user is able to view only the uncoverable portion 520 of the screen. When the door 500 is in the open position, the user is able to view the uncoverable portion 520 and the coverable portion 530 of the screen. Thus, the coverable portion 530 of the screen is prevented from being viewed when the door 500 is in the closed position, as shown in FIG. 5.

The door 500 may cover any portion of the screen when the door is in the closed position. In some examples, no portion of the screen is covered when the door is in the open position. In other examples, only a portion of the screen is covered when the door is in the open position. When the door is in the closed position, information may be displayed on both the coverable portion and the uncoverable portion of the screen. Alternatively, when the door is in the closed position, information may be displayed in only the uncoverable portion of the screen. Any suitable information may be displayed in the coverable and uncoverable portion of the screen when the door is in either the open or closed positions.

In some examples, the medical device further includes an actuator for the user to control one or more operations of the module. When the door is in the closed position, the door may also cover the actuator. The actuator may include a button, dial, or other user control that permits the user to enter data into the module or manipulate data existing in the module to provide treatment and/or monitoring of the patient.

The module of the medical device may contribute or otherwise provides an input that is represented by an image that is projected or displayed in some portion of the screen. The image may be displayed in one or both of the coverable or uncoverable portions of the screen. In a specific example, the image is displayed in the coverable portion of the screen. In this example, the image includes information that assists the user to perform manual defibrillation on the patient. Any information, data, images, or the like displayed or operative in the coverable portion of the screen may include more advanced or complex features and options available on the medical device to help more experienced users to treat and/or monitor a patient. The information, data, images, or the like that are displayed or operative in the uncoverable portion of the screen may include less advanced features and options available on the medical device to aid a less experienced user to treat and/or monitor a patient.

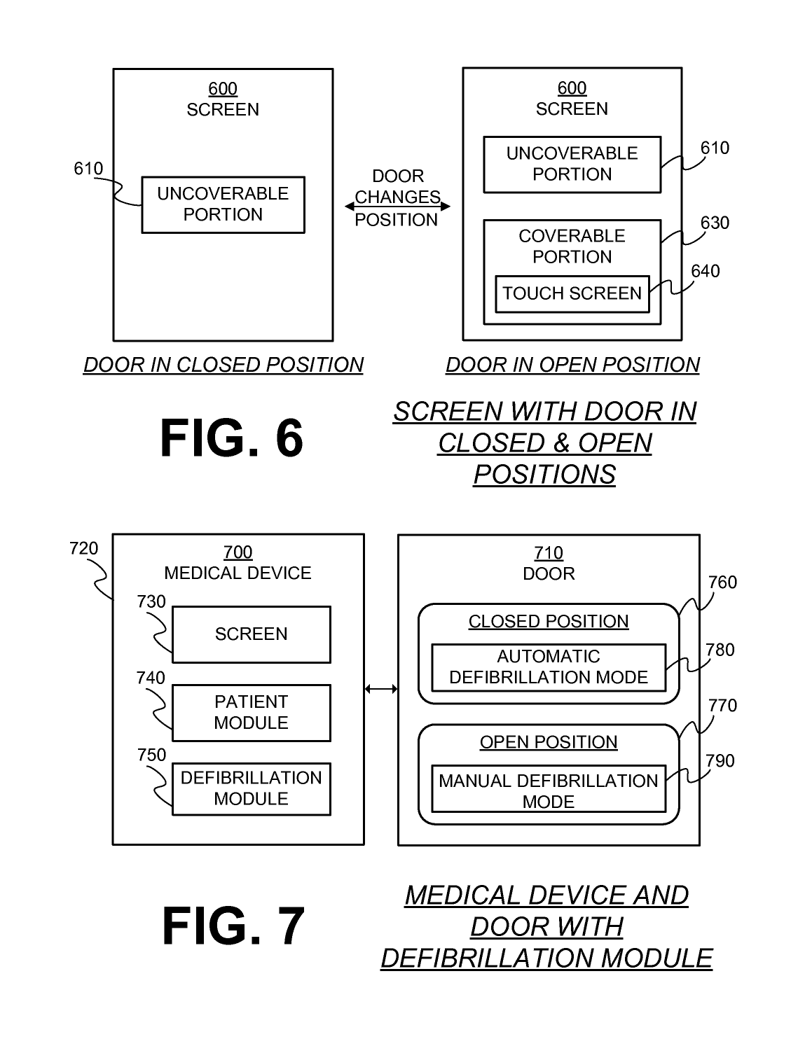

FIG. 6 illustrates the screen 600 of the medical device when the door changes position between the closed position and the open position. When the door is in the closed position, the uncoverable portion 610 of the screen 600 is viewable by the user. The coverable portion 630 is not viewable by the user when the door is in the closed position. When the door is in the open position, both the uncoverable portion 610 and the coverable portion 630 of the screen 600 are viewable by the user. FIG. 6 shows an example medical device in which the coverable portion 630 of the screen 600 includes a touch screen 640. The touch screen 640 is sensitive to touch by the user and may display a soft key for touching by the user. Any suitable touch screen capabilities may be included in this example. The touch screen 640 is structured to receive user input and interact with the medical device in any suitable manner, such as to allow the user to interact with the patient module and optionally any other modules, if present, of the medical device.

The disclosed medical device includes a detector that detects the position of the door. For example, the detector may detect the door in the open position or the closed position. The detector can also detect when the door is not positioned properly in either the open position or the closed position and may optionally issue an alert or otherwise prompt or notify the user to properly position the door in the open or closed position. A processor in the medical device may be responsive to an output from the detector. The processor can operate a first protocol when the door is in the open position and a second protocol when the door is in the closed position. In examples that include a detector, the processor may operate the first protocol when the detector detects the door to be in the open position and may operate the second protocol when the detector detects the door to be in the closed position. The first and second protocol may be different in any suitable manner.

For example, the patient module is a defibrillation module for the medical device or the medical device includes a patient module and a defibrillation module. FIG. 7 shows a medical device 700 having a housing 720, a screen 730, a patient module 740, and a defibrillation module 750. When the door is in the open position 770, the defibrillation module 750 operates in manual defibrillation mode 790. When the door is in the closed position 760, the defibrillation module 750 operates in automatic defibrillation mode 780. In the example medical devices that include a detector and a processor, as described above, when the detector detects the door to be in the closed position, the processor operates the automatic defibrillation mode. When the detector detects the door to be in the open position, the processor operates the manual defibrillation mode. The first and second protocols can be any other suitable protocols and can operate any functions of the medical device.

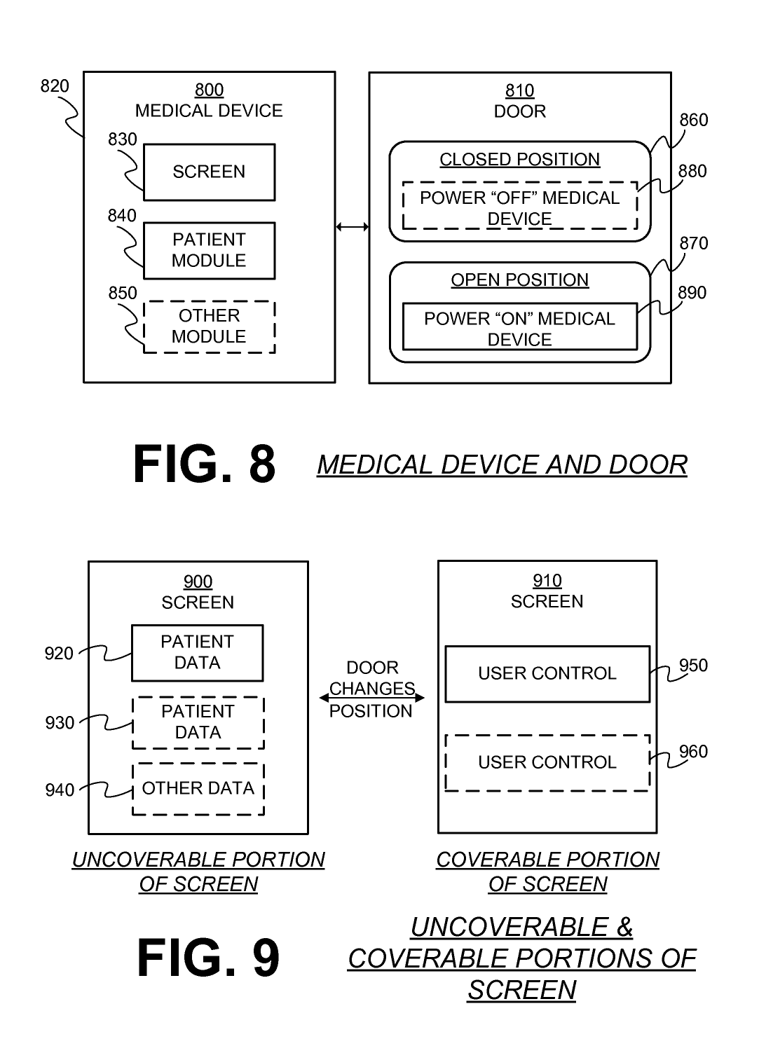

FIG. 8 shows another example medical device 800 with a housing 820, a screen 830 attached to the housing 820, a patient module 840 and optional other modules located within the housing 820, and a door 810 coupled to the housing. In this example, the door controls the operating state of the medical device depending on the door's position. As described above, the door 810 is movable between a closed positioned 860 and an open position 870. In some examples, the medical device is structured to power on when the door is moved to the open position. In other examples, the medical device is structured to power off when the door is moved to the closed position. Still other examples incorporate both the medical device structured to power on when the door is in the open position and the medical device is structured to power off when the door is in the closed position.

In example medical devices that include a defibrillation module, when the door is in the closed position, the medical device powers on user controls associated with the automatic defibrillation mode of the defibrillation module. In other example medical devices that include a defibrillation module, when the door is in the open position, the medical device powers on user controls associated with the manual defibrillation mode of the defibrillation module. Still other example medical devices incorporate both options such that when the door is in the open position, the medical device powers on user controls associated with the manual defibrillation mode and when the door is in the closed position, the medical device powers on user controls associated with the automatic defibrillation mode.

The medical device 800 of FIG. 8 may also power on a portion of the medical device itself or any user controls, such as the defibrillation controls described above, when the door is in the closed position. Such a closed door configuration is useful during transport and monitoring of a patient to prevent inadvertent activation of any functions or user controls of the medical device. Likewise, the medical device 800 may alternatively or additionally power on a greater portion of the medical device, such as additional functions or user controls, when the door is in the open position. Such an open door configuration is useful during treatment and monitoring of the patient to permit the user to access a greater amount of functions and user controls than when the door is in the closed position.

FIG. 9 shows an example of uncoverable 900 and coverable 910 portions of the screen of the medical device that are viewable by the user when the door changes position from the open position to the closed position. Patient data 920, 930 may be displayed in the uncoverable portion of the screen. Any suitable type and amount of patient data may be included. In some examples, only one piece of patient data 920 is displayed in the uncoverable portion of the screen, such as patient's ECG signal. A second piece of patient data 930 may also optionally be displayed, such as the patient's pulse or blood pressure. Other data 940 can optionally be displayed, such as the patient's temperature or other vital signs.

The medical device may include a screen that includes a display having a first portion and a second portion. The first portion of the display is viewable on the screen when the door is in the closed position and the first portion and the second portion of the display are viewable on the screen when the door is in the open position. The first portion of the screen is coverable by the door when the door is in the closed position. The second portion of the screen is always viewable by the user when the door is in either the open or closed position. In some examples, the first portion or coverable portion of the screen includes one or more user controls that are enabled when the door is in the open position. Such user controls may be covered by the door when the door is moved to the closed position. More specifically, in some examples, the user controls in the coverable portion of the screen are inoperative when the door is moved to the closed position. Referring again to FIG. 9, the covered portion of the screen 910 includes at least one user control 950 and optionally a second user control 960. Relating the user control configuration to the defibrillation example described above, the user control(s) may include controls that aid a user in performing manual defibrillation on the patient.

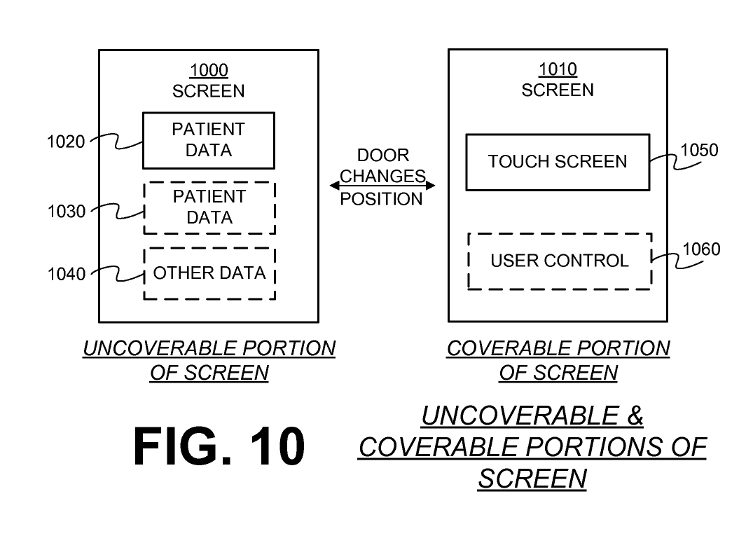

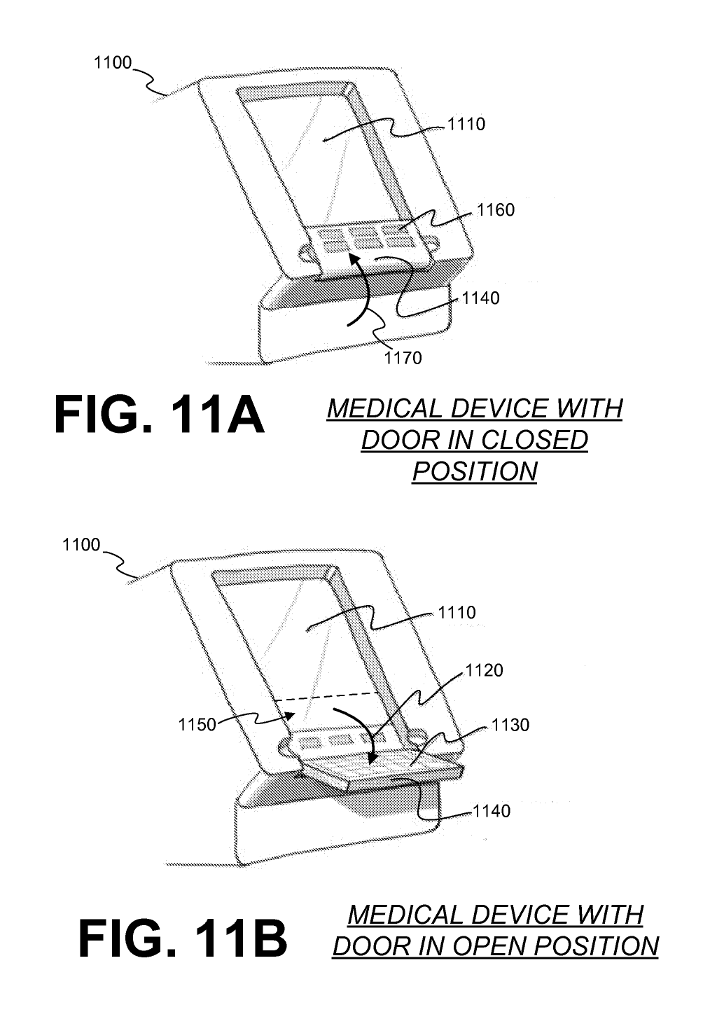

FIG. 10 illustrates an example of uncoverable 1000 and coverable 1010 portions of the screen of the medical device that are viewable by the user when the door changes position from the open position to the closed position. Similar to the configuration of the screen shown in FIG. 9, patient data 1020, 1030 may be displayed in the uncoverable portion of the screen along with any other suitable data 1040. The coverable portion of the screen 1010 includes a touch screen 1050 and optionally a user control 1060, such as any of the user controls described above. The touch screen 1050 may include one or more touch sensitive controls that are operable by the user.

FIGS. 11A and 11B show an exemplary medical device 1100 with the door 1140 in the closed position 1170 and the open position 1120, respectively. The medical device also includes a screen having an uncoverable portion 1110 and a coverable portion 1150. When the door 1140 is in the closed position 1170, multiple user controls 1160 are viewable by and operative for the user to treat and/or monitor a patient. When the door 1140 is in the open position 1120, several user controls 1130 are viewable by and operative for the user. The several user controls 1130 that are available to the user when the door 1140 is in the open position 1120 exceed the number and/or complexity of the user controls 1160 available to the user when the door is in the closed position 1170.

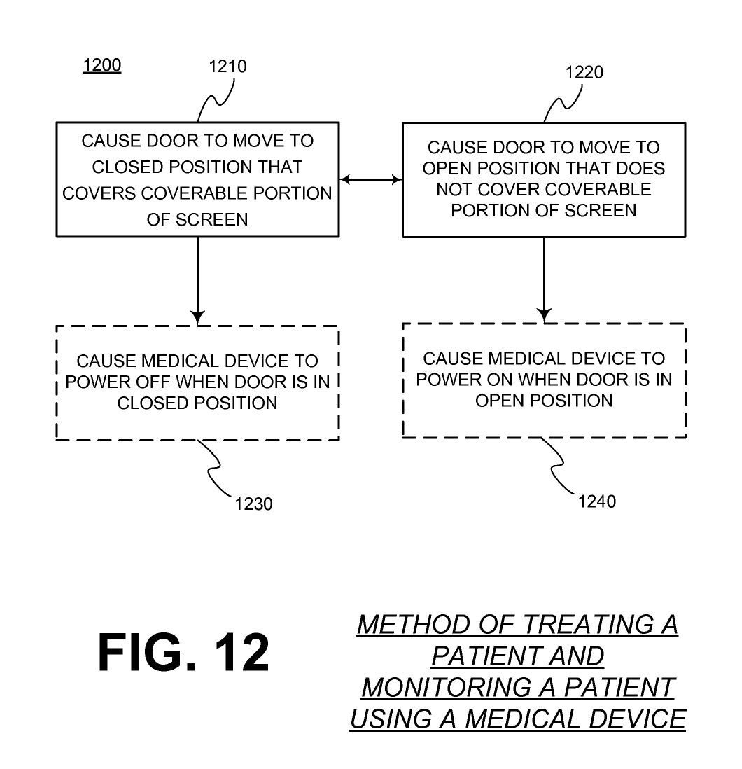

Referring now to FIG. 12, a method 1200 of at least one of treating or monitoring a patient parameter using a medical device is disclosed. The method uses one of the medical devices described above. When the door of the medical device is caused to move to a closed position, the door covers a coverable portion of the screen 1210. When the door of the medical device is caused to move to an open position, the door does not cover the coverable portion of the screen 1220. Optionally, the method also affects the operative state of the medical device or some portion of the medical device. For example, the medical device may be caused to power off when the door is in the closed position 1230 and may be caused to power on when the door is in the open position 1240. Still further various user controls can be enabled or disabled when the door is in the open or closed position, respectively.

In this description, numerous details have been set forth in order to provide a thorough understanding. In other instances, well-known features have not been described in detail in order to not unnecessarily obscure the description.

A person skilled in the art will be able to practice the present invention in view of this description, which is to be taken as a whole. The specific embodiments as disclosed and illustrated herein are not to be considered in a limiting sense. Indeed, it should be readily apparent to those skilled in the art that what is described herein may be modified in numerous ways. Such ways can include equivalents to what is described herein. In addition, the invention may be practiced in combination with other systems.

The following claims define certain combinations and subcombinations of elements, features, steps, and/or functions, which are regarded as novel and non-obvious. Additional claims for other combinations and subcombinations may be presented in this or a related document.

What is claimed is:

1. A medical device for a user to one of treat a patient and monitor a parameter of the patient, comprising:
a housing;
a module located within the housing for the one of treating the patient and monitoring the patient parameter;
a screen attached to the housing, for being viewed by the user outside the housing; and
a door coupled with the housing, the door movable between a closed position that covers at least a coverable portion of the screen to prevent it from being viewed, and an open position that does not cover the coverable portion of the screen.

2. The device of claim 1, in which
when the door is in the closed position, information is displayed in the coverable portion of the screen.

3. The device of claim 1, in which
the module is a defibrillation module,
when the door is in the open position, the defibrillation module operates in manual defibrillation mode, and
when the door is in the closed position, the defibrillation module operates in automatic defibrillation mode.

4. The device of claim 1, in which
the door can be moved between the open position and the closed position by being rotated.

5. The device of claim 1, in which
the door can be moved between the open position and the closed position by being slid.

6. The device of claim 1, in which
when the door is in the open position, no portion of the screen is covered.

7. The device of claim 1, in which
the coverable portion of the screen is sensitive to touch and displays a soft key for touching by the user.

8. The device of claim 1, further comprising:
a detector for detecting whether the door is in the open position or in the closed position; and
a processor for operating, responsive to an output from the detector, a first protocol when the door is the open position and a second protocol when the door is in the closed position.

9. The device of claim 1, further comprising:
an actuator for the user to control an operation of the module, and
in which, when the door is in the closed position, the door also covers the actuator.

10. The device of claim 9, in which
the module contributes an input that is represented by an image, and
at least a portion of the image is projected in the coverable portion of the screen.

11. The device of claim 10, further comprising:
a detector for detecting whether the door is in the open position or in the closed position; and
a processor for operating, responsive to an output from the detector, a first protocol when the door is the open position and a second protocol when the door is in the closed position.

12. The device of claim 11, in which
the module is a defibrillation module,
when the door is in the open position, the defibrillation module operates in manual defibrillation mode, and
when the door is in the closed position, the defibrillation module operates in automatic defibrillation mode.

13. The device of claim 1, in which
the medical device is structured to power on when the door is moved to the open position.

14. The device of claim 13, in which
the medical device is structured to power off when the door is moved to the closed position.

15. The device of claim 1, in which
the coverable portion of the screen includes one or more user controls that are enabled when the door is in the open position.

16. The device of claim 15, in which
the user controls are covered by the door when the door is moved to the closed position.

17. The device of claim 16, in which
the user controls are inoperative when the door is moved to the closed position.

18. The device of claim 1, in which the screen includes a display including a first portion and a second portion, and in which the first portion of the display is viewable on the screen when the door is in the closed position and the first portion and the second portion of the display are viewable on the screen when the door is in the open position.

19. The device of claim 18, in which the first portion of the display shows data associated with at least one of the treatment and monitoring of the patient and the second portion of the user controls includes at least one touch sensitive control.

20. A method of one of treating a patient and monitoring a parameter of the patient using a medical device that includes a housing, a patient module located within the housing for the one of treating the patient and monitoring the patient parameter, a screen attached to the housing for being viewed by the user outside the housing, and a door coupled with the housing, the method comprising:
causing the door to move to a closed position that covers at least a coverable portion of the screen to prevent the coverable portion of the screen from being viewed by the user; and
causing the door to move to an open position that does not cover the coverable portion of the screen.

21. The method of claim 20, further comprising:
causing the medical device to be powered on by positioning the door in the open position.

22. The method of 21, further comprising:
causing the medical device to be powered off by positioning the door in the closed position.

23. The method of claim 1, further comprising:
enabling one or more user controls when the door is in the open position.

24. The method of claim 23, further comprising:
covering the user controls by the door when the door is moved to the closed position.

25. The method of claim 24, further comprising:
causing the user controls to be inoperative when the door is moved to the closed position.

26. The method of claim 1, further comprising:
enabling one or more user controls only when the door is in the open position.

27. The method of claim 1, in which
the door can be moved between the open position and the closed position by being rotated.

28. The method of claim 20, in which
the door can be moved between the open position and the closed position by being slid.
